(19) United States
(12) Patent Application Publication
(10) Pub. No.: US 2005/0150029 A1
(43) Pub. Date: Jul. 14, 2005

(54) Finger guard

(76) Inventor: Earl Votolato, Newport Beach, CA (US)

Correspondence Address: Mr. William W. Haefliger, Suite 512, 201 S. Lake Ave., Pasadena, CA, 91101, US

(21) Appl. No.: 10/755,135

(22) Filed: Jan. 12, 2004

(51) Int. Cl. A61F 9/00

(52) U.S. Cl. 2/16

(57) ABSTRACT

A finger guarding device, in combination a culinary finger guard plate, two finger guards upstanding from a side of the plate and having spacing therebetween to receive a user's middle finger to extend generally parallel to the plate, each finger guard having a finger retention portion overhanging the plate to provide adjustable spaces to receive the user's first and third fingers, between said overhang portion and the plate.

### BACKGROUND OF THE INVENTION

This invention relates generally to finger guards useful, for example, during culinary cutting and dicing of food; and more particularly the invention concerns improvements in such guards especially adapted to different size user fingers, while maintaining finger protection.

Prior finger guards were generally of circular loop shape, so as to be fixed in finger reception area. They were therefore clumsy to use, since user's have different size fingers. Also, prior guards did not readily accommodate to reception of three fingers of a user's hand, since loop shaped guards could not snugly or comfortably accommodate to the different cross sectional areas of first, second and middle fingers, of different user's hands, of different sizes.

### SUMMARY OF THE INVENTION

It is a major object of the invention to provide a significantly improved finger guard device that overcomes the above problems and difficulties of prior devices.

Basically, the invention comprises:
a) a culinary finger guard plate,
b) two finger guards upstanding from a side of the plate and having spacing therebetween to receive a user's middle finger to extend generally parallel to the plate,
c) each finger guard having a finger retention portion overhanging the plate to provide adjustable spaces to receive the user's first and third fingers, between said overhang portion and the plate.

As will be seen, the finger retention portions have terminals spaced from the plate, whereby each guard is supported in cantilever relation to the plate, and has corresponding adjustability in size. Such guard cantilever support, at two locations, allows for adjustability of the space between the guards, to accommodate to snug reception of middle fingers of different sizes.

It is a further object of the invention to provide spaced finger guards, and a plate, of molded plastic material, the guards sufficiently thin to flex toward one another in response to first and third finger sideways pressure exertion.

Typically, the guards and plate are integrally molded, so that the guards can flex relatively toward one another to sidewardly grip the user's middle finger. As will be seen, each guard may have hook shape.

Yet another object is to provide a method of guarding the first, third and middle fingers of a culinary worker's hand, the method including the steps:
a) providing dual, spaced, guards with cantilever support at a base plate,
b) inserting said middle finger into the space formed between the two guards,
c) and also inserting said first and third fingers beneath said finger retention portions.

Another step comprises exerting sideward forces from said first and third fingers against guard portions at opposite sides of said spacing therebetween.

A further step includes providing each guard to have a terminal spaced from the plate, allowing the guard to flex during culinary use.

These and other objects and advantages of the invention, as well as the details of an illustrative embodiment, will be more fully understood from the following specification and drawings, in which:

### DRAWING DESCRIPTION

FIG. 1 is an end view of a device incorporating the invention;

FIG. 2 is a side view of the FIG. 1 device;

FIG. 3 is a bottom plan view of the FIG. 1 device; and

FIGS. 4-7 are views showing use of the device.

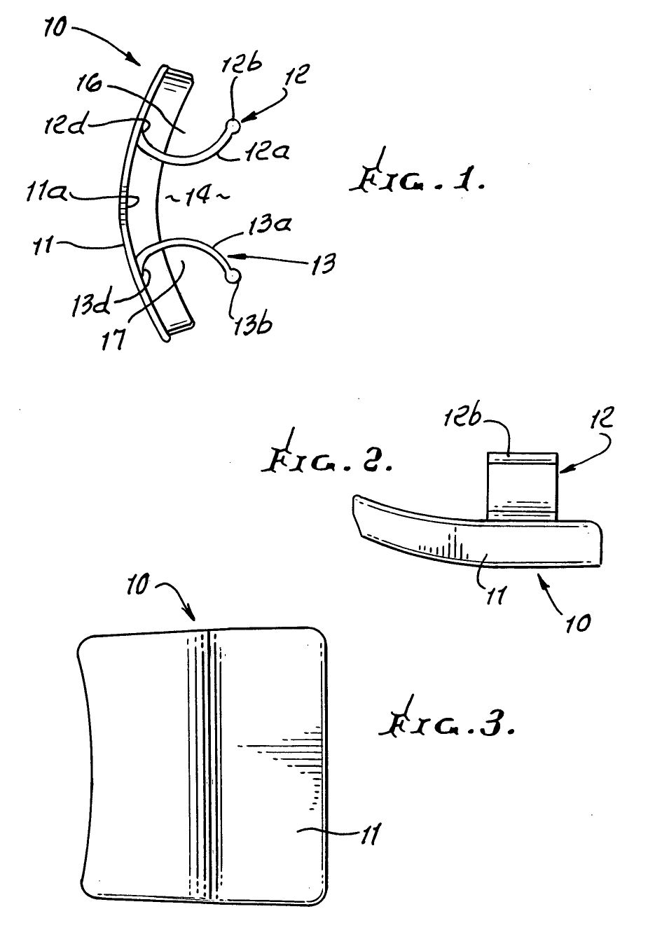

### DETAIL DESCRIPTION

As seen in the drawings, the preferred finger guarding device 10 includes
a) a culinary finger guard plate 11,
b) two finger guards 12 and 13 upstanding from a side 11a of the plate, and having spacing 14 therebetween to receive a user's middle finger 15 to extend adjacent to the plate,
c) each finger guard having a finger retention portion 12a and 13a overhanging the plate to provide for adjustable spaces 16 and 17 to receive the user's first and third fingers, between the overhang portions and the plate.

As shown, the finger retention portions 12a and 13a, are arcuate, and have concave sides facing the plate, each guard having a terminal spaced from the plate, whereby the guard has cantilever support by the plate. See terminals 12b and 13b. See also the plate concave side 11a facing toward the finger guards. The guards have spaced, arcuate projecting portions defining said middle finger space 14 therebetween.

The plate and guards may consist of molded plastic material, and may be sufficiently thin to flex toward one another in response to first and third finger sideways pressure exertion, to adjust the sizes of the finger receiving spaces. Also, the plate and guards are typically integral, at locations of cantilever support of the guards. See such locations at 12d and 13d.

Accordingly, the two guards are configured to flex relatively toward one another to sidewardly grip the user's middle finger. Typically, each guard is a flat strip curved to define a hook shape.

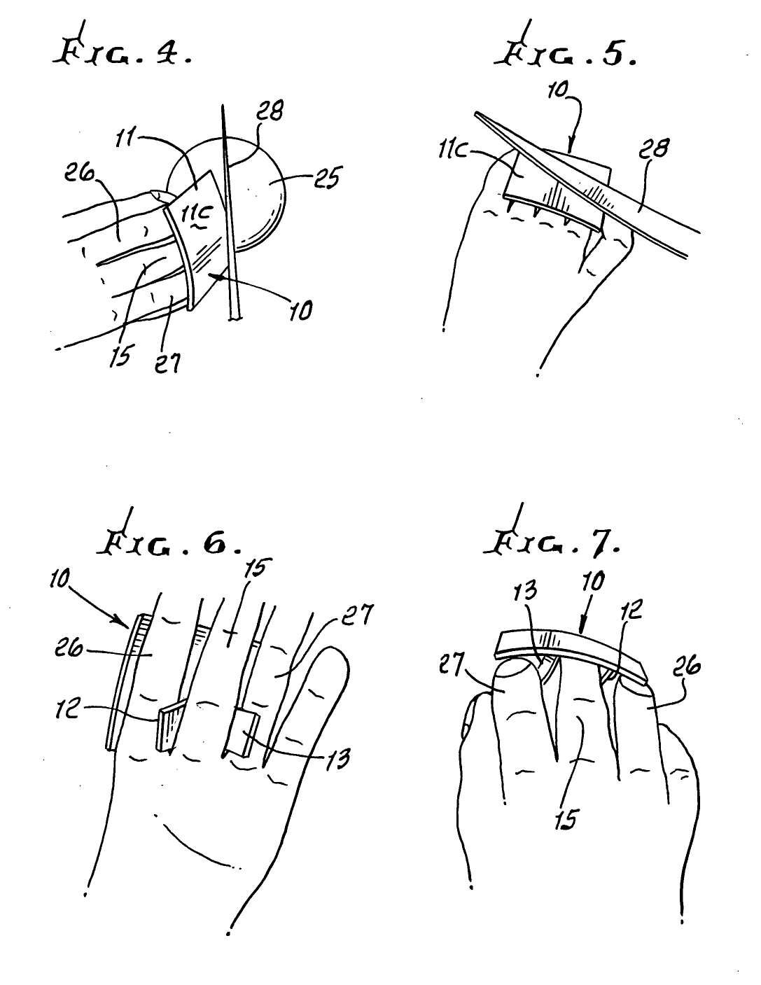

In accordance with the invention, the method of guarding the first, third and middle fingers of a culinary worker, includes the steps,
a) providing a device as described above,
b) inserting the user's middle finger into the space formed between the two guards,
c) and also inserting the first and third fingers into spaces 16 and 17 beneath the referenced finger retention portions,
d) and using the device during culinary cutting of an object or objects, with the bi-directionally convex plate shielding the user's fingers from the cutting tool. See FIGS. 4 and 5. In this regard, the user's thumb and little finger remain free to grip and pick up objects.

Sideward force may be exerted from said first and third fingers against guard portions at opposite sides of the spacing therebetween, to adjust sizes of spaces 16 and 17.

FIGS. 4-7 shows use of the devices, with the user's second, third and fourth fingers inserted in guarded position. FIGS. 4, 5 and 7 show the back side 11c of the plate 11, presented toward a knife 28. FIG. 4 shows cutting of apple 25. First and third fingers are shown at 26 and 27.

The plate and guards may consist of material other than plastic, as for example metal. The plate and guards may be formed separately, and subsequently connected together.

1. A finger guarding device, in combination
a) a culinary finger guard plate,
b) two finger guards upstanding from a side of the plate and having spacing therebetween to receive a user's middle finger to extend generally parallel to the plate,
c) each finger guard having a finger retention portion overhanging the plate to provide adjustable spaces to receive the user's first and third fingers, between said overhang portion and the plate.

2. The combination of claim 1 wherein said finger retention portions are arcuate and have concave sides facing the plate, each guard having a terminal spaced from the plate, whereby each guard has cantilever support by the plate.

3. The combination of claim 1 wherein the plate has a concave side facing toward the finger guards.

4. The combination of claim 2 wherein the plate has a concave side facing toward the finger guards.

5. The combination of claim 1 wherein the guards have generally parallel projecting portions defining said middle finger space therebetween.

6. The combination of claim 1 wherein the plate and guards consist of molded plastic material, the guards sufficiently thin to flex toward one another in response to first and third finger sideways pressure exertion.

7. The combination of claim 6 wherein the plate and guards are integral.

8. The combination of claim 2 wherein the two guards are configured to flex relatively toward one another to sidewardly grip the user's middle finger.

9. The combination of claim 2 wherein each finger guard has hook shape.

10. The method of guarding the first, third and middle fingers of a culinary worker, that includes the steps
a) providing a device as defined in claim 1,
b) inserting said middle finger into the space formed between the two guards,
c) and also inserting said first and third fingers beneath said finger retention portions,
d) and using said device during culinary cutting of an object with said plate shielding the user's fingers from the cutting tool.

11. The method of claim 10 including exerting sideward forces from said first and third fingers against guard portions at opposite sides of said spacing therebetween.

12. The method of claim 1 including providing each guard to have a terminal spaced from the plate, allowing the guard to flex during culinary use.
